(19) United States
(12) Patent Application Publication
(10) Pub. No.: US 2021/0401731 A1
(43) Pub. Date: Dec. 30, 2021

(54) Anti-Aging Formula

(71) Applicant: Marco Ruggiero, Gilbert, AZ (US)

(72) Inventor: Marco Ruggiero, Gilbert, AZ (US)

(21) Appl. No.: 16/911,013

(22) Filed: Jun. 24, 2020

(51) Int. Cl. A61K 8/99; A61Q 19/08; A61K 8/67; A61K 8/9789; A61K 8/34; A61K 8/11; A61K 8/73

(52) U.S. Cl. CPC A61K 8/99; A61Q 19/08; A61K 8/678; A61K 8/73; A61K 8/347; A61K 8/11; A61K 8/9789

(57) ABSTRACT

An anti-aging formula is provided. The anti-aging formula comprises a first microbial blend, a second microbial blend, chondroitin sulfate, vitamin D₃, vitamin E, astragalus, and resveratrol. The first microbial blend comprises one or more lactobacillus bacteria, one or more streptococcus bacteria, one or more bifidobacterium bacteria, and kefir microflora. The second microbial blend comprises one or more lactobacillus bacterium, one or more lactococcus bacteria, and one or more bifidobacterium bacteria. The anti-aging formula is provided as a dietary supplement or drug for human use and is aimed at reversing aging. The formula targets particular genes and proteins that influence the process of aging, including Klotho, telomerase, and sirtuins. In addition, the anti-aging formula induces DNA time dilation, time reversal and quantum entanglement.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to anti-aging formulas and, more particularly, to formulas aimed at reversing aging through targeting genes and proteins known to influence the process of aging and inducing DNA time dilation, time reversal, and quantum entanglement.

### BACKGROUND

Aging is a multifactorial process often marked by a progressive decline in physiological functions. While inevitable, aging, and the characteristics of aging, are undesirable as people usually desire to live as long as possible. The process of aging is influenced by a number of variables that comprise genetic and environmental factors that interact with each other. Three genes, in particular, are especially important determinants for healthy aging because they code for Klotho, telomerase, and sirtuins.

Klotho is a protein with enzymatic activity, including beta-glucuronidase activity, and is involved in several mechanisms associated with healthy aging. For instance, Klotho reduces oxidative stress and inflammation, both of which are major determinants of aging. Klotho is also necessary for maturation of glial cells, myelin integrity, and in this way, protects neurons from toxic effects associated with endogenous or exogenous toxicants that accelerate aging. Moreover, upregulation of Klotho expression has been shown to enhance learning and memory.

In addition, elevated Klotho levels are associated with objective and subjective signs of age reversal. In individuals exhibiting upregulated Klotho expression, healthy life expectancy and cognition are significantly increased. It has also been observed that decreased Klotho expression is associated with accelerated aging and age-related conditions. Overall, upregulation and overexpression of Klotho may extend average life span between 19% and 31%.

Thus, there remains a need for a formula that combines upregulation of gene expression through integrated epigenetic molecular mechanisms and provision of Klotho homologs derived from microbial metabolism in order to increase human Klotho levels.

Telomerase is an enzyme that adds a species-dependent telomere repeat sequence to the 3′ end of telomeres. A telomere is a region of repetitive sequence of DNA at each end of eukaryotic chromosomes. Telomeres protect the end of the chromosome from DNA damage or fusion with adjacent chromosomes. Telomerase maintains the integrity of the linear chromosome structure as a cell divides via mitosis by allowing each cell to replace the lost sequences of DNA at the telomeres. Without telomerase, cells would lose a considerable portion of their chromosomes thereby triggering senescence programs, arresting of cell proliferation, and after a period, apoptosis, or programmed cell death. Telomerase allows the cell to divide without entering into senescence. As a result, upregulation of human telomerase gene expression, and therefore improved maintenance of telomere length, is associated with healthy aging and an increased lifespan.

Sirtuins are a family of signaling proteins that participate in metabolic regulation. The enzymatic actions of sirtuins cause a number of biological effects, including anti-aging and anti-cancer effects. In part, sirtuins regulate fat and glucose metabolism in response to physiological changes in energy levels. Due to this, sirtuins function as crucial regulators of the signaling network that controls energy homeostasis, thereby determining health span.

These three anti-aging determinants, that is, Klotho, telomerase, and sirtuins, are interconnected in a complex integrated molecular crosstalk. As one example, Klotho regulates telomere length and telomerase activity, which is supported by the observation that telomerase activity decreases with Klotho deficiency. Telomerase and sirtuins are also connected. Sirtuins regulate telomere length by a mechanism independent of Klotho and telomerase, and sirtuin overexpression increases telomere length. Moreover, a telomerase deficiency downregulates sirtuin expression and, therefore, telomere shortening functions as an upstream regulator of sirtuins. As a final example, a Klotho deficiency downregulates sirtuin expression and specific sirtuin activators abolish Klotho deficiency-induced arterial stiffness and hypertension. Some formulas and other products exist which aim to target one or more of these pathways. However, no formula exists which targets these three genes or proteins simultaneously and effectively.

### SUMMARY

The present disclosure is directed to anti-aging formulas that reverse aging effects by targeting genes and proteins, including Klotho, telomerase, and sirtuins. More particularly, the formula may upregulate expression of the genes coding for Klotho, telomerase, and sirtuins, all of which provide anti-aging benefits, such as healthy aging and increased lifespan. In addition, the formula may cause DNA time dilation, time reversal, and quantum entanglement, causing further anti-aging effects.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, the anti-aging formula may comprise a first microbial blend, a second microbial blend, chondroitin sulfate, vitamin D₃, vitamin E, astragalus, and resveratrol. In some embodiments, the first microbial blend and the second microbial blend may each comprise one or more lactobacillus bacteria, one or more bifidobacterium bacteria, one or more streptococcus bacteria, and one or more lactococcus bacteria. In addition to the anti-aging effects described herein, each of the aforementioned probiotics of the first microbial blend and the second microbial blend may provide health benefits known to be associated with probiotics, such as balancing bacteria in the digestive system, boosting the immune system, enhancing cardiovascular health, improving mental health.

In accordance with one embodiment, the first microbial blend may comprise one or more lactobacillus bacteria, one or more streptococcus bacteria, one or more bifidobacterium bacteria, and kefir microflora. While a number of lactobacillus bacteria may be provided in accordance with this invention, as a few examples, the one or more lactobacillus bacteria may comprise lactobacillus acidophilus, lactobacillus casei, lactobacillus rhamnosus, lactobacillus reuteri, lactobacillus plantarum, and lactobacillus delbruekii. Similarly, the one or more bifidobacterium bacteria may comprise bifidobacterium bifidum, bifidobacterium lactis, and bifidobacterium breve. In addition, in one embodiment, the one or more streptococcus bacteria may comprise streptococcus thermophilus. A person of ordinary skill in the art will understand that the first microbial blend may comprise other lactobacillus bacteria, streptococcus bacteria, bifidobacterium bacteria, or other types of bacteria.

In certain embodiments, the second microbial blend may comprise one or more lactobacillus bacteria, one or more lactococcus bacteria, and one or more bifidobacterium. The one or more lactobacillus bacteria may comprise lactobacillus salivarius, lactobacillus acidophilus, lactobacillus casei, lactobacillus rhamnosus, or other lactobacillus bacteria. The one or more lactococcus bacteria may comprise lactococcus lactis. In some embodiments, the one or more bifidobacterium of the second microbial blend may comprise bifidobacterium infantis, bifidobacterium bifidum, and bifidobacterium lactis. Of course, one of ordinary skill will recognize other lactobacillus bacteria, bifidobacterium bacteria, and lactococcus bacteria that may be implemented in accordance with this invention.

Taken together, the first microbial blend and the second microbial blend of this formula may synthesize proteins with similarities in structure and function to human Klotho. In this manner, the first microbial blend and the second microbial blend may provide an exogenous source of Klotho and therefore, anti-aging properties.

Chondroitin sulfate may target and favor bioavailability of anti-aging proteins. Specifically, chondroitin sulfate may bind human Klotho and upregulate Klotho, telomerase, and sirtuins. Through microbial metabolism, chondroitin sulfate may form disaccharides that bind to and enhance biological efficacy of vitamins D₃ and E, as well as, astragalus and resveratrol. Chondroitin sulfate may also cause relativistic DNA time dilation and DNA quantum entanglement. Moreover, in some embodiments, chondroitin sulfate may act as a prebiotic.

In some embodiments, vitamin D₃ may upregulate the expression of Klotho, telomerase, and sirtuins. Vitamin D₃ may upregulate Klotho and telomerase expression through association with a vitamin D receptor. On the other hand, vitamin D₃ may upregulate sirtuins via interaction with FoxO proteins.

Vitamin E may also exhibit anti-aging effects may interacting with nuclear receptors in a manner similar to that described with regard to vitamin D₃. Indeed, vitamin E may upregulate expression of Klotho, telomerase, and sirtuins.

In addition, astragalus may activate telomerase, thereby elongated telomeres. Astragalus may also upregulate the expression of Klotho which, in turn, may stimulate telomerase activity. In combination with one or more aforementioned bacteria of the first or second microbial blends, astragalus may further enhance telomerase activity.

Lastly, resveratrol may cause anti-aging effects relating to sirtuins. In accordance with certain embodiments, resveratrol may upregulate expression of Klotho, telomerase, and sirtuins. Resveratrol may also provide an alternative, but complimentary pathway to activate endogenous Klotho production. Further, resveratrol's efficacy may be potentiated by interaction with chondroitin sulfate.

In accordance with some embodiments, the anti-aging formula may be formed as one or more vegetarian capsules. The one or more vegetarian capsules may comprise hypromellose and gellan gum. However, one of ordinary skill in the art will understand that the formula may be provided in other dietary supplement or drug forms.

One or more of the above-disclosed embodiments, in addition to certain alternatives, are provided in further detail below. The disclosed subject matter is not, however, limited to any particular embodiment disclosed.

### Objects and Advantages

Several advantages of one or more aspects are to provide an anti-aging formula that:
(a) Targets genes and proteins that influence the process of aging;
(b) Upregulates expression of genes coding for Klotho, telomerase, and sirtuins;
(c) Allows cell division without senescence;
(d) Prolongs cell apoptosis;
(e) Enhances healthy aging;
(f) Reverses aging;
(g) Increases life span;
(h) Provides known health benefits of probiotics;
(i) Induces DNA time dilation;
(j) Induces time reversal; and
(k) Induces quantum entanglement.

These and other advantages of one or more aspects will become apparent from consideration of the ensuing description. Although the description above contains many specifics, these should not be construed as limiting the scope of the embodiments but as merely providing illustrations of some of several embodiments. Thus, the scope of the embodiments should be determined by the claims that are appended and their legal equivalents, rather than by the examples given. The description of the invention which follows should not be construed as limiting the invention to the examples shown and described, because those skilled in the art to which this invention pertains will be able to devise other forms thereof within the ambit of the appended claims.

### DETAILED DESCRIPTION

Having summarized various aspects of the present disclosure, reference will now be made in detail to certain exemplary embodiments. While the disclosure will be described in connection with these embodiments, there is no intent to limit it to the embodiment or embodiments disclosed herein. Rather, the intent is to cover all alternatives, modifications and equivalents included within the spirit and scope of the disclosure as defined by the appended claims.

An anti-aging formula is provided that, in some embodiments, may reverse the effects of aging. Specifically, the formula may target specific genes and proteins, including Klotho, telomerase, and sirtuins, which may provide anti-aging benefits. Upregulation of Klotho, telomerase, and sirtuins may enhance healthy aging by, for example, allowing cells to divide without senescence and prolonging apoptosis, or programmed cell death. Moreover, the anti-aging formula may induce DNA time dilation, time reversal, and quantum entanglement, further slowing the progression of aging in cells.

In certain embodiments, the anti-aging formula may comprise a first microbial blend, a second microbial blend, chondroitin sulfate, vitamin D₃, vitamin E, astragalus, and resveratrol. The first microbial blend and the second microbial blend may synthesize proteins with similar structures and functions to human Klotho, thereby providing an exogenous source of Klotho. The first and second microbial blends may therefore provide anti-aging effects. The first microbial blend may comprise one or more lactobacillus bacteria, one or more streptococcus bacteria, one or more bifidobacterium bacteria, and kefir microflora. The second microbial blend may comprise one or more lactobacillus bacteria, one or more lactococcus bacteria, and one or more bifidobacterium bacteria.

In some embodiments, the one or more lactobacillus bacteria of the first microbial blend may comprise lactobacillus acidophilus, lactobacillus casei, lactobacillus rhamnosus, lactobacillus reuteri, lactobacillus plantarum, and lactobacillus delbruekii. One or more of the one or more lactobacillus bacteria may enhance telomerase stimulation, thereby causing anti-aging effects. For example, lactobacillus plantarum may synthesize Foldase protein PrsA 2, which may help post-translocational extracellular folding of secreted proteins and interacts with human Src, a non-receptor protein tyrosine kinase.

The percentage weight of the one or more lactobacillus bacteria of the first microbial blend may vary. According to certain embodiments, the one or more lactobacillus bacteria may comprise up to 90.0% weight of the first blend. Moreover, each of the lactobacillus acidophilus, lactobacillus casei, lactobacillus rhamnosus, lactobacillus reuteri, lactobacillus plantarum, and lactobacillus delbruekii may be provided in amounts varying between 5.0% weight to 24.0% weight of the first microbial blend. One of ordinary skill in the art will recognize that each of the one or more lactobacillus bacteria may be provided in other amounts as well. Further, the one or more lactobacillus bacteria may comprise other lactobacillus bacteria not mentioned herein.

The one or more bifidobacterium bacteria may comprise any bifidobacterium bacteria known in the art. In accordance with some embodiments, the one or more bifidobacterium bacteria of the first microbial blend may comprise bifidobacterium bifidum, bifidobacterium lactis, and bifidobacterium breve. In such embodiments, the one or more bifidobacterium bacteria may comprise up to 5.0% weight of the first microbial blend. In addition, each of the bifidobacterium bifidum, bifidobacterium lactis, and bifidobacterium breve may be provided in amounts varying between 1.0% weight to 2.0% weight of the first microbial blend. Of course, the one or more bifidobacterium may be present in other amounts in following this invention.

As mentioned previously, the first microbial blend may further comprise one or more streptococcus bacteria and kefir microflora. The one or more streptococcus bacteria may comprise streptococcus thermophilus. The streptococcus thermophilus may secrete signal peptidase 1, a protein that truncates c-Jun, which may, in turn, enhance the effects of resveratrol. The kefir microflora may be cultured from kefir grains in a lactose-free medium. In certain embodiments, the kefir microflora may comprise up to 1.0% and the one or more streptococcus bacteria may comprise up to 6.0% of the first microbial blend.

According to certain embodiments, each of the bacterium of the one or more lactobacillus bacteria , one or more lactococcus bacteria, and one or more bifidobacterium bacteria may comprise equal percentage weight of the second microbial blend. For instance, each of the bacterium may comprise up to 12.5% weight of the second microbial blend. In other embodiments, the amounts of each of the bacterium may vary and some of the bacterium may be present in quantities proportionately larger than other bacterium.

The one or more lactobacillus bacteria of the second microbial blend may comprise lactobacillus salivarius, lactobacillus acidophilus, lactobacillus casei, and lactobacillus rhamnosus. In alternate embodiments, the one or more lactobacillus bacteria may comprise other types of lactobacillus bacteria . In embodiments where each of the bacterium of the second microbial blend may be present in equal amounts, the aggregate amount of the one or more lactobacillus bacteria may comprise up to 50% weight of the second microbial blend.

The second microbial blend may also comprise one or more lactococcus bacteria. In some embodiments, the one or more lactococcus bacteria may comprise lactococcus lactis. In even further embodiments, the lactococcus lactis may further comprise cremoris as a subspecies. In embodiments where each of the bacterium of the second microbial blend may be present in equal amounts and the one or more lactococcus bacteria may comprise lactococcus lactis, the lactococcus lactis may comprise up to 12.5% weight of the second microbial blend.

The one or more bifidobacterium bacteria of the second microbial blend may comprise bifidobacterium infantis, bifidobacterium bifidum, and bifidobacterium lactis. In alternate embodiments, the second microbial blend may comprise other types of bifidobacterium bacteria. In embodiments where each of the bacterium of the second microbial blend may be present in equal amounts, the one or more bifidobacterium bacteria may comprise up to 37.5% weight of the second microbial blend. The one or more bifidobacterium bacteria may be present in other quantities as well.

Each of the one or more lactobacillus bacteria, bifidobacterium bacteria, streptococcus bacteria, lactococcus bacteria, and kefir microflora of the first microbial blend and the second microbial blend may provide anti-aging benefits. In addition, each of the aforementioned bacteria may provide health benefits known to be associated with probiotics. As an example, the bacteria of the first microbial blend and the second microbial blend may balance bacteria in the digestive system, boost the immune system, enhance cardiovascular health, and improve mental health.

Chondroitin sulfate may have a low molecular weight and may be microbially-derived. In such embodiments where chondroitin sulfate may not be derived from animals, chondroitin sulfate may not contain non-negligible amounts of contaminants found in animal-derived chondroitin sulfate. Indeed, in certain embodiments, chondroitin sulfate may be up to 99% pure and also, may be compatible with vegetarian or vegan diets. Moreover, because microbial-derived chondroitin sulfate may be the result of microbial fermentation, the chondroitin sulfate of this anti-aging formula may be more akin to human chondroitin sulfate.

Chondroitin sulfate may also upregulate Klotho, telomerase, and sirtuins. In some embodiments, chondroitin sulfate may serve as a carrier, favoring bioavailability and targeting of anti-aging proteins. Chondroitin sulfate may form disaccharides through microbial metabolism, which may bind to and enhance biological efficacy of vitamin D₃, vitamin E, astragalus, and resveratrol. In this manner, chondroitin sulfate may enhance the effects of vitamin D₃, vitamin E, astragalus, and resveratrol in upregulating and increasing the activity of Klotho, telomerase, and sirtuins.

According to some embodiments, chondroitin sulfate may be further involved in relativistic DNA time dilation and DNA/tubulin quantum entanglement. As mentioned before, chondroitin sulfate may form disaccharides which may bind DNA and because of the slowing of time, DNA repair mechanism is provided extra time to repair both genetic and epigenetic mutations. As a result, aging may be slowed at the level of DNA. Moreover, chondroitin sulfate may modify quantum properties of DNA which may result in optimizing the ability for DNA to retain, process, and emit information entangled at quantum level.

Vitamin D₃, also known as 1,25-dihydroxyvitamin D or cholecalciferol, may be orally available. Vitamin D₃ may upregulate the expression of Klotho, telomerase, and sirtuins. In certain embodiments, vitamin D₃ may associate with a vitamin D receptor. The ability of vitamin D₃ to interact with the vitamin D receptor may be enhanced by Klotho-like proteins synthesized by the first microbial blend and the second microbial blend. Klotho-like proteins bind the vitamin D₃ carrier, thereby displacing vitamin D₃. Once displaced, vitamin D₃ may bind disaccharides formed through microbial metabolism of chondroitin sulfate. In this way, vitamin D₃ may upregulate Klotho and telomerase expression. Additionally, vitamin D₃ may upregulate sirtuins expression via interaction with FoxO proteins.

Vitamin E may also be orally available and may interact with nuclear receptors in a manner similar to vitamin D₃. In particular, vitamin E may be transported into plasma by a carrier protein, alpha tocopherol transfer protein. The ability of vitamin E to interact with a nuclear receptor may be enhanced by Klotho-like proteins synthesized by the first microbial blend and the second microbial blend. Klotho-like proteins bind the alpha tocopherol transfer protein, thereby displacing vitamin E. Once displaced, vitamin E may bind disaccharides formed through microbial metabolism of chondroitin sulfate.

In some embodiments, astragalus may activate telomerase which may, in turn, elongate telomeres. Astragalus may be orally available and may upregulate expression of Klotho, telomerase, and sirtuins. Astragalus may also upregulate Klotho, thereby further stimulating telomerase activity. In certain embodiments, astragalus may further comprise cycloastragenol. Cycloastragenol may upregulate telomerase by activating signaling pathways. Cycloastragenol may be further enhanced by interaction with chondroitin sulfate and one or more proteins secreted by the one or more bacteria of the first microbial blend and the second microbial blend.

Finally, resveratrol may be provided in the anti-aging formula. Resveratrol is a natural polyphenol. Resveratrol may also be orally available and may cause anti-aging effects relating to sirtuins. In particular, resveratrol may upregulate Klotho, telomerase, and sirtuins expression. More specifically, resveratrol may upregulate expression of Klotho through a c-Jun signaling pathway. In this manner, resveratrol may provide an alternative, yet complimentary pathway to activate endogenous Klotho production. Indeed, resveratrol-mediated transcriptional activation of Klotho may act independently of vitamin D₃, vitamin E, and astragalus.

The anti-aging formula may be formed into any convenient means for human delivery. As one example, the anti-aging formula may be formed as one or more vegetarian capsules. In such embodiments, the one or more vegetarian capsules may comprise hypromellose and gellan gym. The anti-aging formula may be formed into other drug forms, such as tablets. The anti-aging formula may also be formed as a beverage or packaged into a food item. Moreover, the anti-aging formula may be delivered intravenously or via other known means.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### EXAMPLES OF THE PREFERED EMBODIMENTS

In order to more fully teach what the Applicants regards as their invention, the following examples of the certain embodiments of this invention are given. It should be understood that the formulations set forth in these Examples are not to be construed as limiting the scope of the invention, except so far as they comprise an anti-aging formula having the desired properties and characteristics. The following ingredients are an example of embodiments of Applicant's invention with the percentages being given by weight of the composition:

### Example 1: Anti-Aging Formula

| Ingredient | Percentage |
|---|---|
| Vitamin D₃ | 2.60 |
| Vitamin E | 4.89 |
| Chondroitin sulfate | 26.06 |
| First microbial blend | 13.03 |
| Second microbial blend | 13.03 |
| Astragalus | 37.78 |
| Resveratrol | 2.60 |

### Example 2: First Microbial Blend

| Ingredient | Percentage |
|---|---|
| Lactobacillus acidophilus | 14.81 |
| Lactobacillus casei | 14.81 |
| Lactobacillus rhamnosus | 14.81 |
| Lactobacillus reuteri | 14.81 |
| Lactobacillus plantarum | 23.70 |
| Lactobacillus delbruekii | 5.93 |
| Streptococcus thermophilus | 5.93 |
| Bifidobacterium bifidum | 1.48 |
| Bifidobacterium lactis | 1.48 |
| Bifidobacterium breve | 1.48 |
| Kefir | 0.74 |

### Example 3: Second Microbial Blend

| Ingredient | Percentage |
|---|---|
| Lactobacillus salivarius | 12.5 |
| Lactobacillus acidophilus | 12.5 |
| Lactobacillus casei | 12.5 |
| Lactobacillus rhamnosus | 12.5 |
| Lactococcus lactis | 12.5 |
| Bifidobacterium infantis | 12.5 |
| Bifidobacterium bifidum | 12.5 |
| Bifidobacterium lactis | 12.5 |

### CONCLUSIONS, RAMIFICATIONS, AND SCOPE

While certain embodiments of the invention have been illustrated and described, various modifications are contemplated and can be made without departing from the spirit and scope of the invention. For example, various types of lactobacillus bacteria, bifidobacterium bacteria, streptococcus bacteria, and lactococcus bacteria may be used in accordance with this invention. As another example, while the anti-aging formula has been described as being formed into a vegetarian capsule, the anti-aging formula may take virtually any consumable form. Accordingly, it is intended that the invention not be limited, except as by the appended claims.

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the anti-aging formula with which that terminology is associated. In general, the terms used in the following claims should not be constructed to limit the anti-aging formula to the specific embodiments disclosed in the specification unless the above description section explicitly define such terms. Accordingly, the actual scope encompasses not only the disclosed embodiments, but also all equivalent ways of practicing or implementing the disclosed formula. The above description of embodiments of the anti-aging formula is not intended to be exhaustive or limited to the precise form disclosed above or to a particular field of usage.

While specific embodiments of, and examples for, the formula are described above for illustrative purposes, various equivalent modifications are possible for which those skilled in the relevant art will recognize.

While certain aspects of the formula disclosed are presented below in particular claim forms, various aspects of the formula are contemplated in any number of claim forms. Thus, the inventor reserves the right to add additional claims after filing the application to pursue such additional claim forms for other aspects of the anti-aging formula.

What is claimed is:

1. An anti-aging formula, comprising:
a first microbial blend;
a second microbial blend;
chondroitin sulfate;
vitamin D₃;
vitamin E;
astragalus; and
resveratrol.

2. The anti-aging formula of claim 1, wherein the first microbial blend comprises
one or more lactobacillus bacteria;
one or more streptococcus bacteria;
one or more bifidobacterium bacteria; and
kefir microflora.

3. The anti-aging formula of claim 2, wherein
the one or more lactobacillus bacteria comprise
lactobacillus acidophilus;
lactobacillus casei;
lactobacillus rhamnosus;
lactobacillus reuteri;
lactobacillus plantarum;
lactobacillus delbruekii;
the one or more streptococcus bacteria comprise
streptococcus thermophilus;
the one or more bifidobacterium bacteria comprise
bifidobacterium bifidum;
bifidobacterium lactis; and
bifidobacterium breve.

4. The anti-aging formula of claim 3, further comprising
up to 14.8% wt. lactobacillus acidophilus
up to 14.8% wt. lactobacillus casei;
up to 14.8% wt. lactobacillus rhamnosus;
up to 14.8% wt. lactobacillus reuteri;
up to 23.8% wt. lactobacillus plantarum;
up to 5.9% wt. lactobacillus delbruekii;
up to 5.9% wt. streptococcus thermophilus;
up to 1.5% wt. bifidobacterium bifidum;
up to 1.5% wt. bifidobacterium lactis;
up to 1.5% wt. bifidobacterium breve; and
up to 0.75% wt. kefir microflora.

5. The anti-aging formula of claim 1, wherein the second microbial blend comprises
one or more lactobacillus bacterium;
one or more lactococcus bacteria; and
one or more bifidobacterium bacteria.

6. The anti-aging formula of claim 5, wherein
the one or more lactobacillus bacteria comprise
lactobacillus salivarius;
lactobacillus acidophilus;
lactobacillus casei;
lactobacillus rhamnosus;
the one or more lactococcus bacteria comprise
lactococcus lactis;
the one or more bifidobacterium bacteria comprise
bifidobacterium infantis;
bifidobacterium bifidum; and
bifidobacterium lactis.

7. The anti-aging formula of claim 6, further comprising
up to 12.5% wt. lactobacillus salivarius;
up to 12.5% wt. lactobacillus acidophilus;
up to 12.5% wt. lactobacillus casei;
up to 12.5% wt. lactobacillus rhamnosus;
up to 12.5% wt. lactococcus lactis;
up to 12.5% wt. bifidobacterium infantis;
up to 12.5% wt. bifidobacterium bifidum; and
up to 12.5% wt. bifidobacterium lactis.

8. The anti-aging formula of claim 1, wherein the formula is formed as one or more vegetarian capsules.

9. An anti-aging formula, comprising
a first microbial blend comprising one or more lactobacillus bacteria , one or more streptococcus bacteria, one or more bifidobacterium bacteria, and kefir microflora;
a second microbial blend comprising one or more lactobacillus bacteria , one or more lactococcus bacteria, one or more bifidobacterium bacteria;
chondroitin sulfate;
vitamin D₃;
vitamin E;
astragalus; and
resveratrol.

10. The anti-aging formula of claim 9, further comprising
up to 13.1% wt. first microbial blend;
up to 13.1% wt. second microbial blend;
up to 26.1% wt. chondroitin sulfate;
up to 2.6% wt. vitamin D₃;
up to 4.9% wt. vitamin E;
up to 37.8% wt. astragalus; and
up to 2.6% wt. resveratrol.

11. The anti-aging formula of claim 9, wherein the formula is formed as one or more vegetarian capsules.

12. An anti-aging formula, comprising
lactobacillus acidophilus;
lactobacillus casei;
lactobacillus rhamnosus;
lactobacillus reuteri;
lactobacillus plantarum;
lactobacillus delbruekii;
lactobacillus salivarius;
streptococcus thermophilus;
bifidobacterium bifidum;
bifidobacterium lactis;
bifidobacterium breve;
bifidobacterium infantis;
lactococcus lactis;
kefir microflora;
chondroitin sulfate;
vitamin D₃;
vitamin E;
astragalus; and
resveratrol.

13. The anti-aging formula of claim 12, further comprising
up to 3.66% wt. lactobacillus acidophilus;
up to 3.66% wt. lactobacillus casei;
up to 3.66% wt. lactobacillus rhamnosus;
up to 2.62% wt. lactobacillus reuteri;
up to 4.19% wt. lactobacillus plantarum;
up to 1.05% wt. lactobacillus delbruekii;
up to 1.05% wt. lactobacillus salivarius;
up to 1.05% wt. streptococcus thermophilus;
up to 1.31% wt. bifidobacterium bifidum;
up to 1.31% wt. bifidobacterium lactis;
up to 0.26% wt. bifidobacterium breve;
up to 1.05% wt. bifidobacterium infantis;
up to 1.05% wt. lactococcus lactis;
up to 0.13% wt. kefir microflora;
up to 26.10% wt. chondroitin sulfate;
up to 2.60% wt. vitamin D₃;
up to 4.90% wt. vitamin E;
up to 37.80% wt. astragalus; and
up to 2.60% wt. resveratrol.

14. The anti-aging formula of claim 15, wherein the formula is formed as one or more vegetarian capsules.
